(19) United States
(12) Patent Application Publication
(10) Pub. No.: US 2008/0026413 A1
(43) Pub. Date: Jan. 31, 2008

(54) COMPLEXES AND METHODS

(76) Inventor: PHILIP SAVAGE, London (GB)

Correspondence Address: FROMMER LAWRENCE & HAUG, 745 FIFTH AVENUE- 10TH FL., NEW YORK, NY, 10151, US

(21) Appl. No.: 11/758,857

(22) Filed: Jun. 6, 2007

Related U.S. Application Data
(63) Continuation-in-part of application No. PCT/GB05/04725, filed on Dec. 8, 2005.

(30) Foreign Application Priority Data
Dec. 8, 2004 (GB) 0426903.1

(51) Int. Cl. C12N 5/00; G01N 33/00

(52) U.S. Cl. 435/7.92; 435/325; 435/366

(57) ABSTRACT

The invention relates to a cell comprising an exogenous capture moiety on its cell surface, wherein said capture moiety is capable of supporting the attachment of an HLA molecule thereto. In another aspect the invention relates to a cell comprising a capture moiety on its cell surface, and an HLA molecule, wherein said HLA molecule is attached to said cell by means of said capture moiety. Preferably the capture moiety is exogenous, preferably heterologous, prefereably it is CD20. The invention also relates to assays using said cells, and to methods for attaching HLA to them.

### REFERENCE TO RELATED APPLICATIONS

This application is a continuation-in-part of International Patent Application PCT/GB2005/004725 filed October Dec. 8, 2005 and published as WO 2006/061626 on Jun. 15, 2006, which claims priority from Great Britain Patent Application Nos. 0426903.1 filed Dec. 8, 2004.

Each of the above referenced applications, and each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

### FIELD OF THE INVENTION

The invention relates to a system of in vitro diagnostics, and the use of this system. In particular, the invention relates to complexes involving HLA-peptide combinations, their attachment to cells and to the HLA-controlled cells themselves.

### BACKGROUND TO THE INVENTION

Prior art ELISPOT assays have the problem that antigen presenting cells are required to process any antigen and present it to the T cells to elicit T cell activation and cytokine release. These antigen-presenting cells must be matched at all HLA alleles to avoid stimulating a misleading alloreactive response. However it is clearly problematic to have a sufficient range of cloned antigen presenting cells to cover all of the many HLA combinations found in the population.

Another prior art technique is to use artificial or engineered cells as the antigen presenting cells. An example of this is the work of Britten et al. They took K562 cells which are a human CML leukaemia cell line that has no natural HLA class I or class II expression. This cell line was transfected with a single HLA class I allele HLA-A2, to produce a cell K562/A201 that only expresses this allele and no other HLA class I or II molecules. This cell line could then be loaded with binding peptides that are specific for HLA-A2. This produces an antigen presenting cell that only interacts with T cells specific for the allele (HLA-A2) plus the peptide of choice.

The authors reported that when the K562/A201 cells were used, their Elispot assays had similar activity as the commonly used T2 cells but had lower background levels.

According to the state of the art, researchers have to maintain a panel of K562 cell lines each transfected with a different single HLA class I or II gene. However this presents logistical problems in that to cover the population of patient HLA types a large number of different cell lines need to be produced and subsequently kept growing in culture. This presents considerable logistical problems, in terms of keeping multiple cell lines growing and in terms of cross contamination between the different cell lines.

T cell functional assays have been described in the prior art to demonstrate the functional activity of T cells reactive with designated viral or cancer epitopes but have long been a source of difficulty for investigators. These assays are based on the T cell interacting with a target cell that bears the HLA class I (or II) allele plus the appropriate viral, cancer or autoimmune peptide. As a result of the interaction between the T cell receptor of the T cell and the HLA/peptide complex on the target cell the T cell is able to lyse the target cell primarily via the release of toxic enzymes that destroy the target cell membrane. The killing of the target and hence the activity of the T cell can be gauged by release of intracellular contents that can include radiolabelled chromium (⁵¹Cr release assay) or enzyme assays based on the release of LDH or other enzymes from the lysed cells.

One of the main problems with these assays lies with the target cells. Ideally one would like to use the patients own virally infected or tumour cells as these would be completely matched for HLA tissue types and also express the appropriate viral or tumour peptide. However patient tumour cells are rarely available during therapy and frequently are difficult to grow. As a result patient specific tumour cells are impractical for routine use. An alternative is to use that patients own B cells immortalised with the Epstein-Barr virus and use these as target cells when they are peptide pulsed with the appropriate viral/tumour peptide.

Whilst these can be useful for some studies, they have limitations for accurate or large scale use, as an individual cell line needs to be grown for each patient. This is cumbersome and cell lines can not be established from many individuals, and the presence of EBV in the target cells leads to an underlying inaccuracy for all tests and great difficulty with measuring EBV specific activity.

In efforts to overcome these problems other approaches have been examined including transfecting HLA class I negative cells with individual HLA class I alleles to produce specific targets. Whilst this can be of use to specific HLA types, it has not become a routine practice as it presents considerable logistical problems, in terms of keeping multiple cell lines growing and potentially in terms of cross contamination of the different cell lines.

WO 99/64464 is focused on therapy and generation of CTL responses, and concerns Class I HLA in the context of autologous B cells.

The present invention seeks to overcome problem(s) associated with the prior art.

### SUMMARY OF THE INVENTION

As a result of the present invention, the availability of single cell lines, with defined characteristics, which can be used to display individually at the user's discretion any different HLA type and peptide desired leads to considerable practical benefits and cost savings as explained herein.

Assay of CTL activity in the prior art has been hindered by tissue type mismatches and clashes of HLA types in the assay system. Prior art efforts have been strongly directed towards HLA matching, in order to eliminate these conflicts. The present invention is based on the engineering of HLA neutral test cells. In contrast to the prior art HLA matching techniques, the present invention focuses on the construction of cells and complexes for attachment to those cells which bear only the HLA types designed into the system by the user. In this way, the invention advantageously provides a single assay system which is compatible with the assay of CTL's from any HLA background. Since the test system itself contributes no endogenous HLA, conflict or misleading results arising from HLA and mismatch related killing or attack are advantageously reduced or eliminated from the system of the present invention. Thus, the invention relates to cell lines engineered to possess only a single HLA molecule type on the cell surface, and to ELISA and functional assay formats involving such cell lines.

The invention finds particular application in the assay of CTL responses in samples from patients and in the assessment of in vitro based techniques for generating CTL's.

Thus, in one aspect the invention relates to a cell comprising an exogenous capture moiety on its cell surface, wherein said capture moiety is capable of supporting the attachment of an HLA molecule thereto.

In another aspect the invention relates to a cell comprising a capture moiety on its cell surface, and an HLA molecule, wherein said HLA molecule is attached to said cell by means of said capture moiety. In this aspect, the capture moiety may advantageously be endogenous, thereby avoiding the need to manipulate the cell in order to produce expression of the capture moiety. An example of such a cell is a Daudi B cell lymphoma cell which endogenously expresses the CD20 capture moiety to which the HLA molecule may be attached according to the present invention.

In another aspect the invention relates to a cell as described above wherein said capture moiety is exogenous.

In another aspect the invention relates to a cell as described above wherein said capture moiety is heterologous.

In another aspect the invention relates to a cell as described above wherein said capture moiety is CD20.

In another aspect the invention relates to a cell as described above wherein said cell does not express endogenous HLA.

In another aspect the invention relates to a cell as described above wherein said cell is not a naturally occurring antigen presenting cell.

In another aspect the invention relates to a cell as described above wherein said cell is or is derived from human chronic myelogenous leukaemia.

In another aspect the invention relates to a cell as described above wherein said cell is or is derived from human chronic myelogenous leukaemia cell line K562.

In another aspect the invention relates to a complex comprising a cell as described above.

In another aspect the invention relates to a method of attaching a HLA molecule or fragment thereof to a target cell comprising providing the target cell surface with a capture moiety, and incubating the cell with a complex comprising HLA adapted for attachment to said capture moiety. Preferably the capture moiety is CD20.

In another aspect the invention relates to an ELISPOT assay method comprising contacting a cell as described above with a cytotoxic T lymphocyte.

In another aspect the invention relates to a functional T cell assay comprising contacting a cell as described above with a cytotoxic T lymphocyte.

In another aspect the invention relates to use of the complex as described above in an assay as described above.

In another aspect, the invention provides a method of attaching a HLA molecule or fragment thereof to a target cell comprising (i) contacting the cell with an attachment means capable of binding selectively to the capture moiety and (ii) contacting the cell with a complex comprising HLA adapted for binding to said attachment means.

In another aspect, the invention provides a method as described above wherein the capture moiety comprises CD20, the attachment means comprises the B9E9 single chain antibody-streptavidin fusion protein, and the complex comprises biotinylated HLA-class I.

Thus it will be appreciated that overall the invention provides a new system for generating cells for assays, and assays involving such cells. In particular the invention provides a two-step system for attaching a HLA to a cell comprising a first step of contacting the cell with an attachment means, which preferably comprises an antibody or fragment thereof capable of recognising the capture moiety, and subsequently contacting the cell with an HLA adapted to be capable of associating with the attachment means. The attachment means and the HLA preferably each comprise one part of a two-part coupling system for ease of association; to this end, in a preferred embodiment, the attachment means comprises streptavidin and the HLA comprises biotin. The cell may express the capture moiety endogenously for example when the capture moiety is CD20 the cell is preferably a Daudi cell. In other embodiments the cell does not express the capture moiety endogenously and this capture moiety is provided by the present invention for example by transgene expression such as CD20 transgene expression.

Thus it can be seen that the invention advantageously provides a system which can employ one cell (type) to support attachment of numerous different class I and/or class II HLAs (eg. 30-40 different HLAs), and to which HLAs can be bound any peptide of interest (including the 100's of peptides of current medical interest). Thus, advantageously a single cell type together with stable stocks of recombinant HLAs and attachment means can be used to assay almost any clinically relevant HLA, and the peptides of interest can simply be associated with those HLAs before attachment, thereby dramatically simplifying the system compared to prior art assay systems.

Advantageous Features

The prior art drives towards simplification. By contrast, the present invention represents the complication of the system, in particular the provision of the capture moiety on the cell surface. This is prima facie contrary to what is taught in the prior art since it involves considerable extra labour and effort on the part of the operator in order to provide an exogenous capture moiety whereas the prior art conveniently attaches to cell surface proteins which are already present on the cells. However, one of the key advantages of the present invention is by provision of an exogenous capture moiety then HLA conflicts which inhibit prior art assays can advantageously be alleviated. Indeed, in a preferred embodiment of the invention, the only HLA type present in the assay system, in particular on the cells of the assay system, is the HLA type provided by the operator. This is a key advantage of the present invention.

Another advantage of the present invention is in the recognition of the problem in the art. The prior art contains numerous teachings regarding antigen presentation, and a choice of workable systems for accomplishing this. However, HLA conflicts are less problematic in antigen presentation since the key objective in that area is to stimulate responses to that antigen. However, when it comes to assaying of those responses, the present invention provides a significant advantage in that it provides an HLA controlled system of antigen presentation. This system alleviates many or all of the problems which can be associated with the context of the antigen or contributions made by alternative HLA molecules present in the test system. Thus the present invention advantageously allows a much greater degree of control and a much greater elimination of confounding influences when assaying CTL responses compared with prior art systems.

Another key advantage of the present invention is the universal applicability of the system. Prior art systems require individual HLA matched cell lines in order to assay CTL responses for individual HLA type sources. Advantageously, the system according to the present invention is HLA neutral or HLA controlled. Therefore, the same basic system can be applied to the assay of CTL from any HLA typed individual since the HLA type in the assay system is specified and controlled by the operator. Therefore, significant savings in terms of costs and effort in maintaining numerous different HLA matched cell lines are advantageously avoided by use of the present invention. Furthermore, reproducibility and cross comparison of results is enhanced by the common core of the assay system which can be applied to the assay of CTL from such a diverse range of subjects, which is another advantage of the present invention.

The prior art has focused on B cells comprising HLA such as patients' own B cells. It is an advantage of the present invention that B cells with no HLA (eg. with no HLA class I and/or with no HLA class II, preferably with no HLA at all ie. no class I and no class II HLA) are used to create cells with only the desired HLA on their surface for optimal assays.

### DETAILED DESCRIPTION OF THE INVENTION

Capture Moiety

The term “capture moiety” as used herein refers to molecule on the cell surface to which the HLA molecule binds, preferably through intermediates such as the attachment means. The capture moiety may be any cell surface molecule which can be bound by an antibody eg. any cell surface antigen (CSA). Preferably the capture moiety is a B cell marker. Preferably the capture moiety is CD19 or CD20, preferably CD20.

Preferably the capture moiety is stable on the cell surface. By ‘stable’ is meant that it is not recycled/shed/internalised so quickly as to interfere with the assay. In other words, ‘stable’ means that the capture moiety will persist on the cell surface for a period of time which allows the assay to be completed. Preferably stable means that the capture moiety persists for at least 8 hours, eg. 8 hours after attachment of the HLA. Preferably the capture moiety persists for at least 3 days, preferably at least 4 days from attachment. CD20 is a preferred stable capture moiety.

In some aspects, the capture moiety may advantageously be endogenous, thereby avoiding the need to manipulate the cell in order to produce expression of the capture moiety. An example a cell suitable for this aspect of the invention is a Daudi B cell lymphoma cell which endogenously expresses the CD20 capture moiety to which the HLA molecule may be attached according to the present invention

Preferably the capture moiety is a molecule which is not naturally occurring on that cell. Preferably the capture moiety is heterologous. Even more preferably the capture moiety is exogenous. Most preferably the capture moiety is present due to transgene expression. Transgene expression may be transient for example through transgene transfection, or may be stable for example through stably transfected cell lines. Preferably the transfection is stable transfection and preferably the transgene is stably incorporated into the cell's genetic material.

The capture moiety can be added to the cell or caused to be expressed by the cell, and is preferably caused to be expressed by the cell. In the context of being expressed, the capture moiety may be expressed from a naturally silent gene in the genetic material of the cell in question, or may be expressed from exogenous nucleic acid. The nucleic acid may be introduced into the cell by any suitable means such as transfection. Transfection may be transient or stable. Preferably transfection is stable.

Preferably the capture moiety is not naturally expressed in the starting cell type. Thus provision of the capture moiety may be by manipulation of gene expression to activate the gene of interest. Preferably the capture moiety is exogenous. Exogenous has its natural meaning ie. arising from a source outside the organism or cell. Clearly this may still be manufactured inside the cell eg. by expression of exogenous nucleic acid. Preferably the capture moiety is not encoded by nucleic acid naturally found in the starting cell's genetic material, preferably not present in that genome, preferably not present in the source organism's genome. Thus preferably the capture moiety is heterologous. Preferably the capture moiety is provided by expression of a transgene. Preferably this transgene is transfected into the cell. Preferably this transgene is stably transfected into the cell.

Examples of preferred capture moieties according to the present invention include cell surface antigens, cell determinant molecules, or other cell surface borne entities. Preferably the capture moiety is CD20 or CD19, preferably CD20.

Attachment Means

The attachment means is a molecule which selectively binds to the capture moiety. One part of the attachment means is associated with the HLA-peptide complex, and the other part of the attachment means is the part which selectively binds the capture moiety. The attachment means may be any suitable molecule which is capable of binding the capture moiety and also associating with the HLA-peptide complex. Preferably association with the HLA-peptide complex is by chemical bonding, preferably by hydrogen bonding, more preferably by covalent bonding.

Exemplary attachment means include antibodies or fragments of antibodies, or fusions thereof, or sfvSA to the capture moiety. Preferably the attachment means comprises sfvSA to CD20. Preferably the attachment means comprises the B9E9 single chain antibody/streptavidin fusion protein (sfvSA).

In a highly preferred embodiment, the attachment means and HLA molecule may be part of a single covalently linked molecule. Even more preferably this single covalently linked molecule further comprises the recognition peptide (target peptide).

HLA/Peptide Complex

The HLA molecule (such as a HLA class I or class II molecule) or fragment thereof may bind a peptide, which peptide is arranged to be presented for T cell recognition by said HLA molecule or fragment thereof. Said peptide may be attached to the HLA molecule or fragment thereof in accordance with the method described in Garboczi (PNAS 89 1992, 3429-3433).

The attaching means preferably comprises a linking polypeptide with high specific affinity for the capture moiety on the surface of the target cell. The capture moiety may be any molecule such as a cell surface molecule but is preferably a polypeptide based molecule. Capture moiety may be: carcinoembiyonic antigen, placental alkaline phosphatase, polymorphic epithelial mucin, human chorionic gonadotrophin, CD19, CD20, prostate specific antigen, ca-125, HMW-MAA and others, preferably CD20.

Conveniently, the linking polypeptide will comprise an antibody, preferably a monoclonal antibody, capable of reacting/binding with said capture moiety (Riethmuller and Johnson, Curr. Opin. Immunol. 4, 1992, 647-655). Suitable antibodies for this purpose include C46, 85A12, H17E2, HMFGI, W14, 1F5, 225.28s (Buraggi 1985 Cancer Res. 45. 3378-3387), and others. Deposits of the immortalised hybrids producing these antibodies have been made at the American Type Culture Collection, Rockville Md., USA. Further examples of antibodies are described in Maloney et al (Blood 84, 1994, 2457-2466), Riethmuiler et al (Lancet 343, 1994, 1177-1183) and Hird et al (Br. J. Cancer 68, 1993, 403406).

Said linking polypeptide may comprise an antibody raised against a capture moiety and a coupling system for coupling said antibody to said HLA class I molecule or fragment thereof. The coupling system may comprise a two- or three-step chain of well-characterised paired small molecules, joined to the antibody and the HLA class I molecule so as to form a stable bridge between the two. Examples of paired small molecules which might be used in this connection include (but are not limited to) biotin and avidin/streptavidin (Moro, 1997 Cancer Res. 57, 1922-1928; Altman et al, Science 274, 1996, 9496), and calmodulin and calmodulin binding peptides (Neri, 1996, J. Invest. Dermatol. 107, 164-170). Alternatively, said linking polypeptide may comprise an antibody-raised against a capture moiety, which antibody is adapted to be attached directly to said HLA class I molecule or fragment thereof.

In a further possible embodiment of the invention, said complex may comprise a recombinant protein, which recombinant protein includes a moiety comprising said HLA molecule or fragment thereof, and a moiety comprising said attaching means. The HLA molecule or fragment thereof may be purified from plasma or platelets or made recombinantly. The HLA molecule or fragment thereof may further be arranged to bind and present for T cell recognition a defined peptide of choice, such as a viral, bacterial, parasitic, or tumour-specific peptide. Attachment of the HLA molecule or fragment thereof to the capture moiety may be achieved by introducing said HLA molecule or fragment thereof and said attaching means to the vicinity of the capture moiety on the target cell. The target cell may be a culture cell in vitro, but will advantageously be originally taken from the body of a patient. Preferably, the target cell will be arranged to be contacted by a cytotoxic T cell, and if that cytotoxic T cell is adapted to recognise said HLA molecule or fragment thereof in the context of the peptide bound thereto, then this will generate a read-out in the ELISPOT or functional assays of the invention.

The HLA/peptide complex for use in the invention may be any HLA/peptide complex that is of immununological interest. Preferably, the HLA is a class I or II HLA, preferably class I. When it is a class I HLA, preferably the HLA comprises one or more of HLA-A1, HLA-A2, HLA-A3 or HLA-B7. In a preferred embodiment, the HLA is HLA-A2. Specific examples of HLA/peptide complexes include, but are not limited to, HLA class I/telomerase (pan tumor), HLA-A2/melan A (melanoma), HLA-A2/WT1 (leukaemia), or any other peptides of interest.

Attachment

The attachment means is capable of selectively binding to the capture moiety, and to the HLA/peptide complex. Preferably the complex is attached to the cell by attachment means comprising a molecule capable of selective binding to the capture moiety. Preferably the attachment means comprises sfvSA to CD20 or CD 19. Preferably the attachment means comprises sfvSA to CD20, such as the B9E9 moiety.

HLA

The choice of HLA molecule is a matter for the operator. HLA molecules and their sequences are well known in the art. In particular, it should be noted that invention embraces the use of Class I and/or Class II HLA, or a combination thereof. It is an advantage of the present invention that the HLA type in the system is governed entirely by operator choice, and is not constrained by the source material or other factors.

A further advantage of the present invention is that it avoids the need for multiple transfections. Prior art techniques require an individual transfection to be performed for every single HLA type which is required. By contrast, the present invention advantageously provides a single cell line to which any HLA type can be attached merely by exogenously adding the complex and bringing it into contact with the cells. This increases reproducibility since the cell line is always constant, and since the complexes can be pre-prepared and simply added at the time of the assay. The whole transfection and verification process can be advantageously omitted according to the present invention. Furthermore, this significantly decreases preparation times and decreases sample processing times by avoidance of the lengthy transfection step.

Preferably attachment of HLA to the cell is via a system comprising an antibody or antibody fragment.

Preferably attachment of the HLA is to a HLA class I and Class II negative cell line (such as a CD20 transfected K562). These cells are then preferably used for functional assays as mono-specific CTL targets or in Elispots using them as mono-specific antigen presenting cells as described below.

The present invention finds application in diagnostics. In a preferred embodiment the invention relates to engineered K562 cells.

Cells

Basal or starting cells ie. before HLA attachment preferably will have one or more characteristics selected from;
1/ HLA class I negative
2/ HLA class II negative
3/ EBV negative

Preferably the cells have two or more, preferably all three of said characteristics.

In preferred aspects, the present invention then prepares the cells for attachment of HLA by providing a suitable capture moiety for attaching recombinant HLA class I or II complexes to the starting cells.

Preferably the starting cells are K562 cells. These are a human myeloid leukaemia cell line that expresses no HLA class I or II molecules and does not bear the EBV virus.

Preferably the capture moiety is provided by stably transfecting the cells with a gene, preferably for human CD20.

In some embodiments, the cells preferably possess the further characteristic of endogenously expressing a capture moiety, preferably CD20. The advantage of this embodiment is saving labour which would otherwise have to be expended in provision of the capture moiety by transfection or similar approach, and avoidance of any effects on the cells associated with such treatments. A preferred cell of this embodiment is a Daudi cell which expresses class II HLA and has EBV infection, but is advantageously class I HLA negative and expresses the CD20 capture moiety endogenously.

Further Components

Once the cells of the invention are prepared (as necessary) in this manner, optionally further components are attached. Preferably an antibody delivery system, such as that based on the B9E9 single chain antibody-streptavidin fusion protein (sfv-SA) is used to attach streptavidin to the CD20 on the surface of the cells (eg K562-CD20 cells as above).

Advantageously, recombinant biotinylated HLA class I or HLA class II complexes could be stably attached to these cells. Preferably using the biotin streptavidin system.

It is an advantage of the present invention that the choice of recombinant HLA molecule class and allele and peptide give the functional identity to the cell and so allow interaction only with the T cell of choice.

Mono-Specific HLA Coated Cells

The invention provides a method to improve Elispot and/or T cell functional in vitro assays.

The accurate monitoring and enumeration of endogenous T cell responses and those resulting from vaccine therapies in patients with a number of disease particularly cancer and HIV is becoming an increasingly important area.

However the methodologies to do these assays are relatively insensitive, of limited reproducibility and can be cumbersome in view of the wide range of HLA types in patient populations.

Approaches that improve the sensitivity of these assays, make them of increased reproducibility and avoid the use of differing target cell lines for patients of differing HLA types are needed in the field.

The present invention provides a significant step forward in the accuracy and value of in vitro T cell testing relative to prior art techniques.

In a preferred embodiment the assays are performed as follows;

1/ Elispot

This assay examines the production of cytokines, particularly interferon gamma and granzyme B, in response to antigen exposure. T cells are cultured along with antigen presenting cells in wells coated with an antibody to the cytokine of interest. After incubation the cells and culture fluid are washed off and the presence of any secreted cytokine/granzyme is then detected by the binding of a further specific antibody combined with an enzyme assay based detection system.

Preferred peptides are viral (CMV, EBV, influenza) or cancer (Melan-A) peptides.

By detecting the number of spots on the bottom of the plate an estimate of the number of T cells specific for a particular antigen is made.

These cells advantageously have no other HLA molecules that could give rise to non-specific T cell activation and so inappropriate spots in the Elispot assay.

Similar to the situation with Elispot assays described above, the availability of single cell line, with defined characteristics, which can be used to display individually at the investigators discretion every different HLA type and peptide desired leads to considerable practical benefits and cost savings according to the present invention.

The HLA mono Specific Antigen Presenting Cell or Target Cell

One aim the present invention is improving the accuracy of assays.

The invention provides a new approach—that of using HLA mono-specific cells combined with an attaching means for recombinant HLA complexes. These cells find application as either the antigen presenting cell for Elispot analysis and/or as target cells for functional assay.

Further Advantages of This System

HLA monospecific APCs/targets can be made, simply, rapidly and reproducibly to any chosen HLA class I or II allele combined with any peptide of choice.

Only one cell line needs to be kept growing in culture for use irrespective of the HLA types that the user may be wishing to examine.

The cell line (or lines) has the same baseline characteristics, whenever/wherever it is used and with any choice of HLA/peptide complex.

The absence of any other HLA complexes on the surface of the HLA mono-specific cell allows for reduction in non-specific signal that can limit the efficiency of these assays in prior art systems.

These cells reduce the complexity associated with Elispot/functional assays, improve the quality of the data and are simple, quick and reliable to use.

Elispot and T Cell Functional Assay Methodology

1/ Elispot

The description of an increasing number of T-cell defined viral and tumor antigens has led to a rapidly increasing number of antigen-specific vaccination trials designed to treat patients with chronic viral infections or cancer.

However, despite the description and identification of these antigens, there is relatively little information about their immunogenicity in patients, and the optimal methods to vaccinate.

One of the most important requirements for any effective vaccine system is the ability to measure the quantity and quality of any T cell responses produced during the course of immunization. This is at the core of the present invention.

One of the most frequently used approaches to measure T cell function and number is the ELISPOT assay that allows a direct quantification of single T cells based on their rapid cytokine secretion upon antigen contact.

As a result of their high sensitivity and practicability, IFN-gamma ELISPOT assays and more recently granzyme B assays are widely used to monitor antigen-specific T cell immune responses in patients during immunotherapy trials. The invention applies equally to either assay embodiment.

At present there is little standardisation of protocols for frequency analysis with ELISPOT. While some laboratories apply non-fractionated PBMC, others prefer to use purified T cell sub-populations that are seeded with a defined number of antigen-presenting cells.

In total PBMC, the absolute numbers of antigen-specific T lymphocytes and APC can vary in individual blood samples that are collected at several time points during the vaccination course. Additionally the autologous APCs produced on differing occasions might differ in the expression of HLA, costimulatory and adhesion molecules, thereby impeding comparative frequency analyses of antigen-specific T lymphocytes in different individuals. This could restrict the comparability of results obtained from different non-fractionated PBMC samples in a single patient. In support of this view, a multi-centre comparative study performed in four European laboratories suggested the superior sensitivity of an IFN-gamma ELISPOT assay when purified CD8q T cells, rather than non-fractionated PBMC (Scheibenbogen et al., 2000).

Cells of the present invention are preferably used in assays according to Scheibenbogen, C., Romero, P., Rivoltini, L., Herr, W., Schmittel, A., Cerottini, J., Wolfel, T., Eggermont, A. M., Keilholz, U., (2000) “Quantitation of antigen-reactive T cells in peripheral blood by IFN-gamma-ELISPOT assay and chromium-release assay: a four-centre comparative trial.” J. Immunol. Methods 244, 81, which is incorporated herein by reference.

However ELISPOT assays on purified CD4 and CD8 T cell sub-populations, require the use of exogenous antigen presenting cells to stimulate the T cells in the assay.

These can be autologous (ie the patients own) or allogeneic HLA-matched APC. However because of the limited size of patients' blood samples, the patient's own autologous APC are rarely available in sufficient numbers to perform these assays. Therefore, the use of a common allogeneic peptide presenting cell line appears advantageous. One widely used allogeneic -APC for HLA-A2-restricted CD8 T lymphocytes is T2 cell line, which can be very efficiently loaded with exogenous HLA-A2-binding peptides.

However, the T2 cell line whilst bearing the HLA-A2 complex also has other HLA class I and II complexes some of which will be an HLA mismatch with the patients own HLA types. This underlying alloreactive interaction can result in ELISPOT assays producing a strong background CD8 T lymphocyte reactivity against the HLA mismatches on the T2 cells. As a result of these alloreactive immune responses in some HLA-A2 individuals can prevent the detection of low frequency T cell responses.

The human cell line K562 was originally established from the pleural effusion of a female patient with chronic myelogenous leukaemia CML. K562 cells lack HLA classes I and II expressions on their cell surface.

It has previously been demonstrated that K562 cells transfected with the gene for HLA-A2 are low background inducing APC that can efficiently present HLA-A2-binding peptides to CD8 T lymphocytes in IFN-gamma ELISPOT assays (Britten C M, Meyer R G, Kreer T, Drexler I, Wolfel T, Herr W. The use of HLA-A*0201-transfected K562 as standard antigen-presenting cells for CD8(+) T lymphocytes in IFN-gamma ELISPOT assays. J Immunol Methods. Jan. 1, 2002; 259(1-2):95-1; incorporated herein by reference).

Whilst the results with the HLA-A2 transfected K562 cells appear to give an improvement over those obtainable with T2 cells, the applicability of these cells is limited to just the HLA class I allele transfected.

To expand the choice of alleles, the cDNA to all of the common HLA class I and class II alleles are available and a series of cell lines can be constructed to include all the alleles of choice. However this results in a requirement for large number of cells lines to be kept in culture and also the possibility of contamination between the cell lines.

According to the present invention, HLA-neutral (ie. HLA negative) cells are equipped with a capture moiety to which the desired HLA complex can be attached in vitro eg. using recombinant HLA complex. Thus, by the provision of a single ‘universal’ cell line according to the present invention the problems of the prior art multi-cell-line and multi-transfection approaches are alleviated.

An advantage of the system of the invention is that it provides an approach to use an antibody delivery system to deliver recombinant HLA complexes to be attached to cell surface molecules (capture moieties) on the surface of the target cells.

By attaching HLA complexes to cells as described herein, the production of target cells that can successfully and specifically interact with T cells recognising the added complex is enabled.

These cells are especially useful in assays such as ELISPOT and functional assays such as Cr-release assays which represent advantageous embodiments of the present invention.

T Cell Functional Assays

The activity of T cells can be gauged by release of intracellular contents that can include radiolabelled chromium (⁵¹Cr release assay) or enzyme assays based on the release of LDH or other enzymes from the lysed cells.

At present these are rarely included in routine monitoring of T cell function in response to vaccine treatments. However, the present invention enables them to become of widespread application.

There are a number of reasons for this;
1/ The relatively low level of reactive T cells in the PBMC population
2/ The difficulty performing the assays
3/ The lack of reproducibility for the assay.

The choice of target cells for this assay can include;
1/ Autologous tumour cells
2/ Peptide pulsed autologous B cells
3/ HLA defined target cells such as native T2 cells
4/ gene transfected CIR-A2 cells that are naturally HLA class I negative but carry the HLA-A2 cDNA so express only a single HLA allele.

There are drawbacks with each of these approaches.
1/ Autologous tumour cells, are rarely routinely available and are usually difficult to grow.
2/ Peptide pulsed autologous B cells, are hard to grow, are individualised for each patient, and carry the EBV virus that can lead to misleading results from natural EBV responses.
3/ T2 cells are HLA-A2+ve but also carry other HLA alleles that can give misleading results from alloreactive interactions between the T cells and the other alleles. There are relatively few cell lines like this, which are well characterised for their tissue type. Additionally the T2 cell line carries the EBV, which is a source of misleading results and also of contamination to other cell lines.
4/ Gene transfected cell lines. These have the problem of needing a different transfectant for each HLA allele. This presents the difficulties of keeping a large number of cell lines in culture and the risk of inadvertent cross-contamination.

The present invention advantageously addresses these problems by the use of a single defined cell line that has no natural HLA class I or II molecules and is able to be used for any HLA molecule.

Advantages include:

Only one cell line needs to be kept in culture

The cell has no natural HLA class I or II molecules and so elicits no alloreactive responses

The cells can be prepared to the same standard method and quality for repeat experiments and also be comparable between centres.

### BRIEF DESCRIPTION OF THE FIGURES

FIG. 1 shows a diagram of interaction of mono-specific HLA cell with T cell (ie. a diagrammatic view of Mono-specific HLA coated cells)

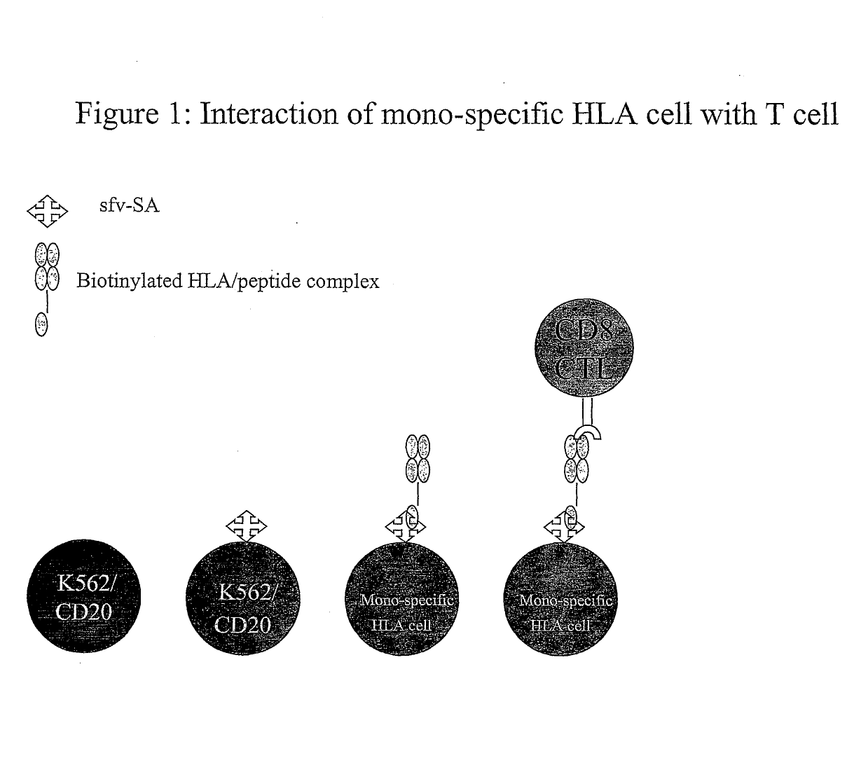

FIG. 2 shows a diagram of production of a range of standardized mono-specific allele/peptide CTL targets for functional assays (ie. an example of a functional assay using mono-specific HLA coated cells).

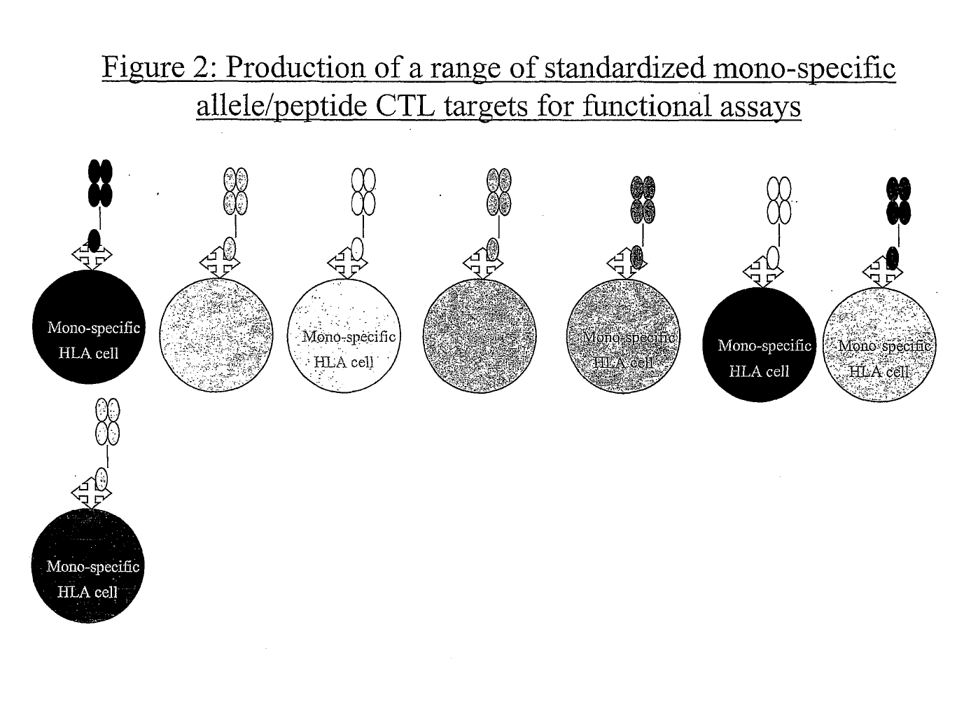

FIG. 3 shows a bar chart of activity of HLA-A2/Melan-A specific CTLs against mono-specific HLA target cells

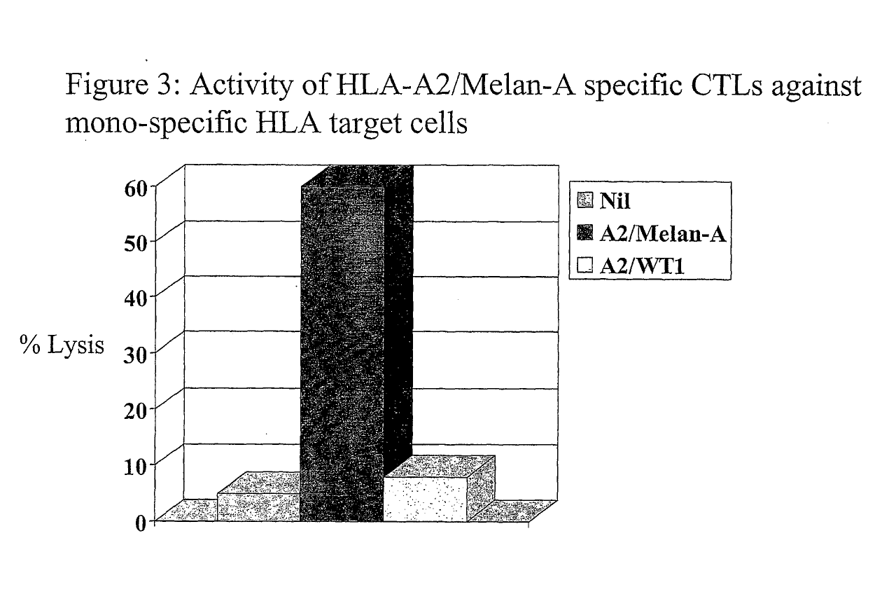

FIG. 4 shows a diagram of mono-specific HLA cells as APCs in Elispot assay

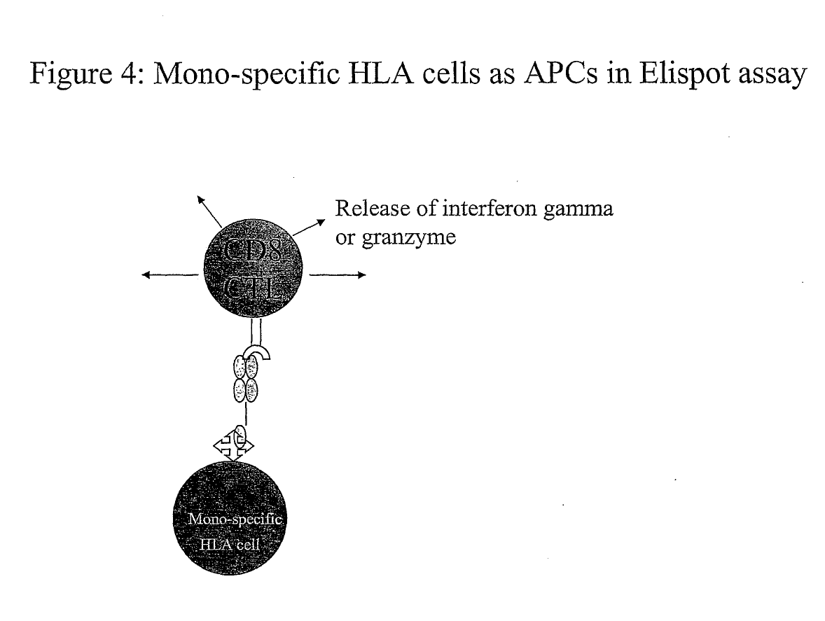

FIG. 5 shows intracellular cytokine analysis

### EXAMPLES

### Example 1

### Making Cells

In this example, cells according to the present invention are made.

The starting cells are K562 cells.

The capture moiety is CD20.

Nucleic acid encoding CD20 cloned into a gene expression construct capable of driving expression of CD20 in K562 cells.

This expression construct is transfected into K562 cells.

Stable transfectants are selected.

Cell surface expression of the CD20 capture moiety is confirmed using anti-CD20 antibodies.

### Example 1a

### Manufacture of Complexes

The cells of example 1a are expanded by culture in vitro.

B9E9 single chain antibody-streptavidin fusion protein sfvSA B9E9 is incubated with the cells and the excess washed away.

Biotinylated HLA-class I bearing the Melan-A peptide is incubated with the cells and the excess washed away.

Thus a complex according to the present invention is made.

### Example 2

### ELISPOT

Applying this technology to the Elispot environment is done in the following way.

A HLA class I and class II negative cell line (such as K562)

Transfect with the gene for capture moiety such as human CD20 (or another antigen stably expressed on the cell surface)

Use an antibody to the capture moiety bearing streptavidin or biotin (either chemically or recombinantly attached) to attach to the capture moiety (eg. cell surface antigen).

Sequentially attach an HLA class I or II complex (joined chemically or recombinantly to biotin/streptavidin).

This system allows the production of a wide range of mono-specific HLA class I or II targets to any desired allele/peptide complex. Some benefits of this are;
A/ The mono-specific cells will have near identical characteristics when used on different occasions.
B/ Only a single cell line will need to be kept in culture, which can be used with any allele
C/ As the cells are otherwise HLA class I and II negative there should be no alloreactive activity and hence background Elispot activity should be very low
D/ The mono-specific HLA class I/II cells have a relatively high and uniform epitope density that should give good and reproducible levels of T cell interaction and so good Elispot results.

### Example 3

### Functional T Cell Assay

Below is standard description for how a functional T cell assay is performed (often termed a chromium release assay)

CIR-A2 cells were labelled with 2 uCi/uL of 51Cr (Amersham Pharmacia, UK) for 1 h at 37 C then washed. CIR-A2 cells were pulsed with the peptide of choice at a concentration of 10 uM for 1 h at 37 C. The target cells were plated at 3000 cells per well in U bottomed 96-well plates. PBMCs, media or 5% Triton X-100 were added to a final volume of 200 ml. Plates were incubated for 4 h at 37 C in a 5% CO2 atmosphere and 50 ml of supernatant was collected and added to 150 ml of scintillant.

The specific lysis was calculated as:

$$\% lysis . \frac{experimental cpm - spontaneous cpm}{maximum cpm - spontaneous cpm} : 100 \%$$

The spontaneous release was measured from the cells incubated in media alone, the maximum release was measured from the cells incubated in 5% Triton.

Each target should only be lysed by CTL of the individual allele/peptide specificity

This example illustrates the easy change in identity of targets provided by the present invention.

Furthermore, the wide choice of allele/peptide combinations available commercially are each suitable for use in the assays of the present invention

It can be appreciated that only one cell line needs to be kept in culture.

Standardised targets are advantageously comparable between assays and indeed between assays performed in different centres.

Intracellular cytokine staining data further indicate that this is an effective assay according to the present invention.

### Example 4

### Intracellular Cytokine Assay

The measurement of intracellular cytokine production in response to exposure to antigen presenting cells is a frequently used measure of T cell activity. The present invention facilitates this assay as demonstrated herein.

The use of HLA class I mono-specific cells advantageously allows assays to be more reproducible and with lower cross reactivity.

Here we demonstrate the results of using HLA class I mono-specific cells according to the present invention as the Antigen Presenting Cells in an intracellular cytokine assay.

The Peripheral Blood Mononuclear Cells (PBMCs) used in this example contain an expanded population recognizing the cytomegalovirus (CMV) epitope HLA-A2/NLV.

These PBMC cells were then incubated with;
1/ No additional cells
2/ HLA class I-ve B cells
3/ HLA class I mono-specific B cells bearing HLA-A2/NLV

The HLA class I-ve B cells of this example are Daudi B cell lymphoma cells. These cells have no endogenous HLA class I and express CD 20 naturally. CD20 is the capture moiety in this example.

HLA class I mono-specific B cells bearing HLA-A2/NLV of this example are the same Daudi B cell lymphoma cells. The HLA-A2/NLV complex has been attached according to the methods of the present invention. In this example the Daudi B cell lymphoma cells were contacted with the anti-CD20 antibody B9E9-streptavidin fusion protein as the attachment means. This binds to the capture moiety CD20 which is endogenously expressed in the cells of this example. The complex comprising HLA is a biotinylated HLA monomer. In this example the HLA-A2 monomers are premixed with the NLV peptide and the resulting complex is contacted with the cells. The complex becomes attached by binding of the biotin component of the HLA complex to the streptavidin component of the attachment means. Thus the HLA class I mono-specific B cells bearing HLA-A2/NLV are made.

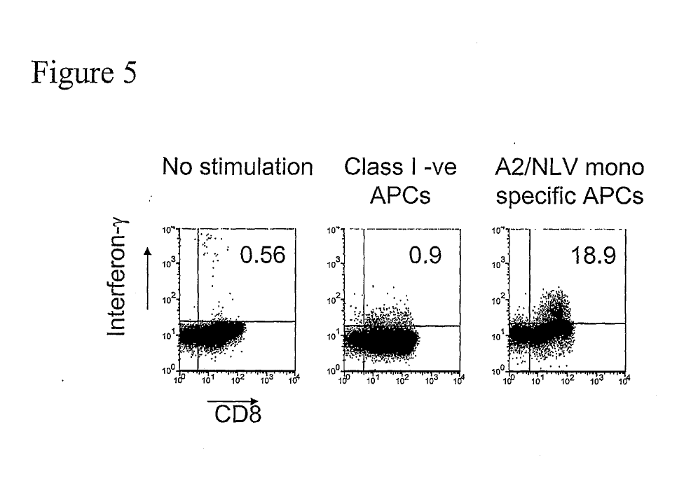

FIG. 5 shows the activity of the PBMCs as assessed by intracellular cytokine analysis.

The results demonstrate that PBMCs which have no additional stimulation show 0.56% positivity on intracellular cytokine staining, cells exposed to the HLA class I-ve B cells show almost no significant increase in intracellular cytokine production 0.9%. However PBMCs that are stimulated with the HLA class I mono-specific B cells bearing the HLA-A2/NLV complexes according to the present invention show 18.9% of the PBMCs to become positive for intracellular cytokine production.

The conclusion to this is that the HLA class I mono-specific B cells give a very precise signal to the PBMCs in this assay, so producing a very clean and simple assay.

All publications mentioned in the above specification are herein incorporated by reference. Various modifications and variations of the described methods, complexes and cells of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.
A. A cell comprising an exogenous capture moiety on its cell surface, wherein said capture moiety is capable of supporting the attachment of an HLA molecule thereto.
B. A cell comprising a capture moiety on its cell surface, and an HLA molecule, wherein said HLA molecule is attached to said cell by means of said capture moiety.
C. A cell according to paragraph A or paragraph B wherein said capture moiety is exogenous.
D. A cell according to any of paragraphs A to C wherein said capture moiety is heterologous.
E. A cell according to any of paragraphs A to D wherein said capture moiety is CD20.
F. A cell according to any of paragraphs A to E wherein said cell does not express endogenous HLA.
G. A cell according to any of paragraphs A to F wherein said cell is not a naturally occurring antigen presenting cell.
H. A cell according to any previous paragraph wherein said cell is or is derived from human chronic myelogenous leukaemia.
I. A cell according to paragraph H wherein said cell is or is derived from human chronic myelogenous leukaemia cell line K562.
J. A complex comprising a cell according to any of paragraphs A to 1.
K. A method of attaching a HLA molecule or fragment thereof to a target cell comprising providing the target cell surface with a capture moiety, and incubating the cell with a complex comprising HLA adapted for attachment to said capture moiety.
L. A method according to paragraph K wherein the capture moiety is CD20.
M. An ELISPOT assay method comprising contacting a cell according to any of paragraphs A to I with a cytotoxic T lymphocyte.
N. A functional T cell assay comprising contacting a cell according to any of paragraphs A to I with a cytotoxic T lymphocyte.
O. Use of the complex according to paragraph J in an assay according to paragraph M or N.
P. A method of attaching a HLA molecule or fragment thereof to a target cell comprising (i) contacting the cell with an attachment means capable of binding selectively to the capture moiety and (ii) contacting the cell with a complex comprising HLA adapted for binding to said attachment means.
Q. A method according to paragraph P wherein the capture moiety comprises CD20, the attachment means comprises the B9E9 single chain antibody-streptavidin fusion protein, and the complex comprises biotinylated HLA-class 1.

1. A cell comprising:
a) an exogenous capture moiety on its cell surface, wherein said capture moiety is capable of supporting the attachment of an HLA molecule thereto; or
b) a capture moiety on its cell surface, and an HLA molecule, wherein said HLA molecule is attached to said cell by means of said capture moiety.

2. A cell according to claim 1 wherein said capture moiety is exogenous.

3. A cell according to claim 1 wherein said capture moiety is heterologous.

4. A cell according to claim 1 wherein said capture moiety is CD20.

5. A cell according to claim 1 wherein said cell does not express endogenous HLA.

6. A cell according to claim 1 wherein said cell is not a naturally occurring antigen presenting cell.

7. A cell according to claim 1 wherein said cell is or is derived from human chronic myelogenous leukaemia.

8. A cell according to claim 7 wherein said cell is or is derived from human chronic myelogenous leukaemia cell line K562.

9. A complex comprising a cell according to claim 1.

10. A method of attaching a HLA molecule or fragment thereof to a target cell comprising providing the target cell surface with a capture moiety, and incubating the cell with a complex comprising HLA adapted for attachment to said capture moiety.

11. A method according to claim 10 wherein the capture moiety is CD20.

12. An ELISPOT assay method comprising contacting a cell according to claim 1 with a cytotoxic T lymphocyte.

13. A functional T cell assay comprising contacting a cell according to claim 1 with a cytotoxic T lymphocyte.

14. Use of a complex comprising a cell according to claim 1 in an assay selected from:
an ELISPOT assay method comprising contacting a cell according to claim 1 with a cytotoxic T lymphocyte, and
a functional T cell assay comprising contacting a cell according to claim 1 with a cytotoxic T lymphocyte.

15. A method of attaching a HLA molecule or fragment thereof to a target cell comprising (i) contacting the cell with an attachment means capable of binding selectively to the capture moiety and (ii) contacting the cell with a complex comprising HLA adapted for binding to said attachment means.

16. A method according to claim 15 wherein the capture moiety comprises CD20, the attachment means comprises the B9E9 single chain antibody-streptavidin fusion protein, and the complex comprises biotinylated HLA-class 1.
